(19) United States
(12) Patent Application Publication
(10) Pub. No.: US 2022/0023090 A1
(43) Pub. Date: Jan. 27, 2022

(54) SANITARY URINATION DEVICE

(71) Applicant: Roy Clair, Lititz, PA (US)

(72) Inventor: Roy Clair, Lititz, PA (US)

(21) Appl. No.: 16/935,756

(22) Filed: Jul. 22, 2020

(51) Int. Cl. A61F 5/451; A61F 5/453; A61F 5/455

(52) U.S. Cl. CPC A61F 5/451; A61F 2005/4402; A61F 5/4556; A61F 5/453

(57) ABSTRACT

A biodegradable urination-aiding device and method of using the same allows a user to urinate into a receptacle, such as a toilet bowl, from a standing position by passing urine directly into the water contained in a receptacle. The device is manipulatable or configurable from a storage configuration, wherein the device is efficiently stored either by itself, or with a plurality of like-devices, to a use configuration, wherein a body of the device defines an elongated, hollow structure. In use, a user associates a first open end of the device with his or her urethra, and places a second open end of the device into the water contained within the receptacle. With the device associated with the urethra, a user urinates into the device, and urine is conveyed into the receptacle in a clean and quiet manner.

### FIELD OF THE INVENTION

The present disclosure relates to the field of personal hygiene, and more specifically, to a disposable device to aid a male or a female in urinating from a standing position into a toilet in a sanitary manner

### BACKGROUND

Men and women are faced with many challenges using rest facilities, both in their homes and while traveling. For example, men and women often encounter unsanitary toilet conditions, wherein a surface of the toilet must be cleaned prior to use, or in the least, must be handled by a user in the case of lowering or lifting a seat or seat cover prior to use.

In order to avoid these exposures, urinating from a standing position may be preferred. To this end, devices have been developed which enable women to stand while urinating, as often performed by their male counterparts. However, such devices have been tailored to enable use at a urinal, rather than at a toilet. The distinction is significant, as it can be difficult to control the trajectory of urine when urinating from a standing position into a toilet, as the urine must typically travel farther. Urinating from a standing position also increases the risk of excess splashing, and thus the above-described unsanitary toilet conditions, as well as increases the noise resulting from expelled urine contacting the toilet water. All of this problems may be further exaggerated when attempting to urinate in darkness, and/or when tired, such as when one has recently awoken from sleep.

In an effort to mitigate these drawbacks, men will often attempt to direct the flow of their urine onto an exposed sidewall of a toilet bowl in an effort to reduce such splashing and/or noise. However, this behavior can lead to urine being accidentally deposited on other surfaces of the toilet, such as the rim of the bowl, the seat or the floor surrounding the toilet.

For all of the above reasons, improved solutions for enabling easy, discrete and sanitary standing urination for both men and women is desired.

### SUMMARY

Embodiments of the present disclosure include devices for aiding a user in urinating into a receptacle (e.g., a toilet) from a standing position with improved sanitation. Generally, embodiments include an elongated, hollow body formed from a water soluble material and are configured to convey urine from a first open end thereof to a second open end thereof. The body of the device may include a generally continuous and uniform diameter, or may be tapered between its ends, with an inlet or input end opening being larger in diameter compared to an outlet or output end. In one embodiment, the device defines a pleated body foldable in an accordion-like manner, wherein the body is lengthened from a storage configuration to a use configuration by a user prior to urination. In still other embodiments, the device is stored in one of a plurality of rolled manners.

According to another embodiment, a method for aiding a user in urinating into a receptacle from a standing position with improved sanitation is provided. The method includes a first step of configuring or manipulating a water-soluble urination device from a storage configuration, into a use configuration wherein the device defines an elongated hollow body. Once configured, a first open end of the device is associated with a urethra of the user, and a second open end of the device is placed into water contained within the receptacle. In this way, subsequent urination by the user into the device will transfer urine through the device and directly into the water, reducing unsanitary splashing and unnecessary noise. The device may be disposed of into the receptacle after a single use, wherein it will subsequently biodegrade in a safe manner.

Other objects and advantages of the present invention will be apparent by the following description with reference to the accompanying drawings and will be helpful for a comprehensive understanding to the present invention.

### BRIEF DESCRIPTION OF THE DRAWINGS

The above and other features of the disclosure will become more apparent by describing in detail exemplary embodiments thereof with reference to the accompanying drawings, in which:

FIG. 1 is a simplified side view of a urination device in a state of use according to an embodiment of the present disclosure;

FIG. 2 is a side perspective view of a urination device according to the embodiment of FIG. 1;

FIG. 3 is a side perspective view of a urination device according to another embodiment of the present disclosure;

FIG. 4 is perspective view of a plurality of interconnected urination devices packaged according to an embodiment of the present disclosure;

FIG. 5 is a side perspective view of a urination device according to embodiments of the present disclosure in a packaged or collapsed state or configuration;

FIG. 6A is a side perspective view of a urination device according to an embodiment of the present disclosure in a partially-expanded state or configuration;

FIG. 6B is a side perspective view of the urination device of FIG. 6A in a collapsed or packaged state or configuration; and

FIG. 7 is a top view of a toilet fitted with an accessory guide for use with a urination device according to an embodiment of the present disclosure.

### DETAILED DESCRIPTION OF THE EMBODIMENTS

It is to be understood that the figures and descriptions of the present invention have been simplified to illustrate elements that are relevant for a clear understanding of the present invention, while eliminating, for purposes of clarity, many other elements found in, for example, known systems. However, because such elements are well known in the art, and because they do not facilitate a better understanding of the present invention, a discussion of such elements is not provided herein. The disclosure herein is directed to all such variations and modifications known to those skilled in the art.

In the following detailed description, reference is made to the accompanying drawings that show, by way of illustration, specific embodiments in which the invention may be practiced. It is to be understood that the various embodiments of the invention, although different, are not necessarily mutually exclusive. Furthermore, a particular feature, structure, or characteristic described herein in connection with one embodiment may be implemented within other embodiments without departing from the scope of the invention. In addition, it is to be understood that the location or arrangement of individual elements within each disclosed embodiment may be modified without departing from the scope of the invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout several views.

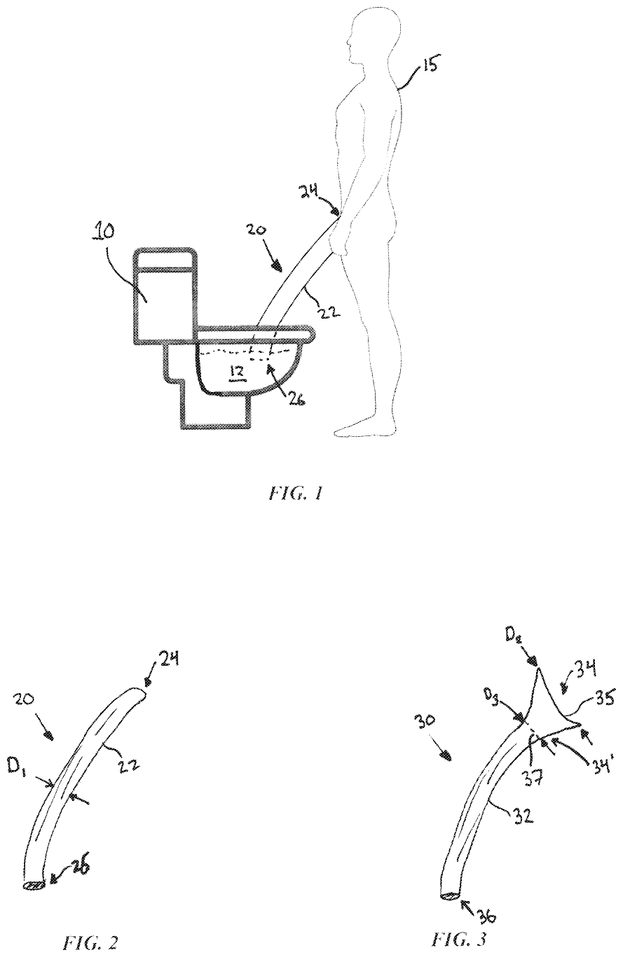

Referring generally to FIG. 1, a urination device 20 according to an embodiment of the present disclosure is used to convey fluid (e.g. urine) from an elevated position directly into a bowl of a toilet 10 (or other receptacle), or into the water 12 filling the toilet bowl. The device 10 defines a closed fluid conduit or funnel, including an elongated, hollow body 22 defining a first open input or inlet end 24 configured to be placed into contact with, or in close proximity to, the urethra of a male or female user 15. The body 22 further includes a second open output or outlet end 26, opposite the first input end 24, and configured to be placed into the bowl of a toilet, and more preferably, into the water 12 contained within the bowl. In one embodiment of the present disclosure, the first input end 24 of the body 22 is sized so as to accommodate the penis of a male user therein, and in others, the body is sized to fit generally to the public area of a man or a woman, as will be set forth in greater detail herein. The device 20 may be formed from, for example, paper or other water-soluble material such that it may be disposed of in the toilet after use. In one embodiment, the paper may include a multi-layer or multi-ply paper, improving durability and stiffness during use. In some embodiments, the paper or other material may be coated so as to temporarily reduce water solubility (e.g., at least for an average duration of urination) before beginning to biodegrade in a manner suitable for disposal into the toilet. Each of the embodiments of the present disclosure may be pre-formed (i.e., sized) with a generally curved profile extending between the input end 24 and the output end 26, or may be formed generally linearly, with gravity and/or the weight of a fluid traveling therethrough introducing the illustrated curvature.

Referring to FIG. 2, in one particular advantageous embodiment, the elongated body 22 of the device 20 includes a generally constant diameter D₁ from the input end 24 to the output end 26. In this way, a plurality of devices may be manufactured as a single, continuous structure, increasing manufacturing ease, as well as ease of packaging and use, as will be set forth in greater detail herein. This embodiment may be best suited for use with a male, as the diameter D₁ may be selected to correspond to, or be marginally larger than, that of an average diameter of a male penis, such that is may be inserted into the input end 24 of the device 20 during urination.

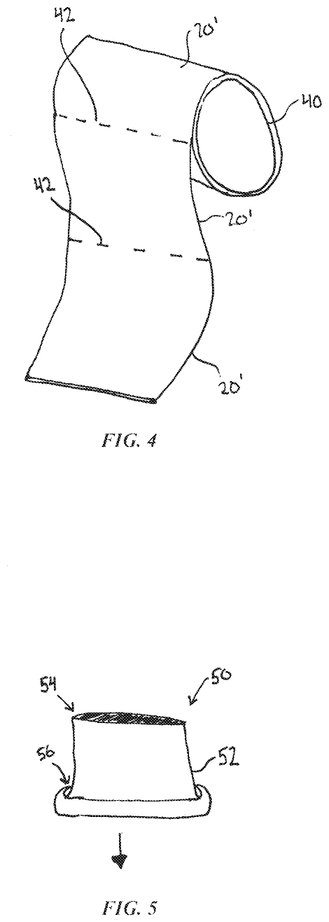

According another embodiment, as shown in FIG. 3, a device 30 may be configured for use by a man or by a woman. As illustrated, a hollow body 32 of the device 30 includes an input end portion having a profile suitable for engaging with the female anatomy (and/or a male anatomy). Specifically, top portion of the body 32 may include a first open input end 34 having an opening of a wider diameter D₂ compared to that of another portion of the body. The first open input end 34 may further define a concave or otherwise contoured circumferential edge 35 suitable for more closely mating with a pubic region of a user. This profile improves the fit or seal of the device 30, reducing the chance of urine leaking from the input end 34 of the device during use. The body 32 may taper gradually from the first input end 34 to a diameter less than that of the input end 34 at an output end 36 thereof.

Still referring to FIG. 3, the contoured top portion of the device 30 including the first input end 34 may be removable. Specifically, perforations 37 may be formed through the body 32 in a direction transverse to a direction of elongation of the body for facilitating the removal of the top portion of the device 30. Below the perforations 37 is a second portion having an outer diameter D₃ which is less than the diameter D₂ of the top portion, and sized at least to accommodate male anatomy. In this way, if a male user prefers, he may remove the top portion of the body 32 by tearing the body along perforations 37, thereby creating a second input end 34′, and utilize the remainder of the body 32 in the manner described above with respect to the embodiment of FIGS. 1 and 2. Likewise, the body 32 may include a visual indication (e.g., a line) of a suitable location for separation, wherein a user may simply tear away the top portion from the remainder of the body at the level of the indicator.

A plurality of any of the devices 20,30 shown in FIGS. 1-3 may be formed from a continuous piece of material, and devices may or may not be separated from one another in advance of their use. Specifically, during manufacturing, a piece of material (e.g, an elongated section of multi-layer coated paper) may be folded along its length, and free edges thereof secured together via, for example, an adhesive to create a hollow body. The resulting elongated structure is readily flattenable, foldable, and/or rollable, offering a plurality of packaging options.

For example, FIG. 4 illustrates an embodiment of the present disclosure wherein a plurality of interconnected devices 20′ (or segments thereof), are wound onto a cylindrical carrier roll 40 for particularly efficient and familiar packaging. Before or after the above-described folding and securing the longitudinal ends of the body material together, a plurality of perforations or perforated lines 42 may be formed transversely through the material, with a spacing therebetween defining the length of the device (e.g., 18-36 inches). In this way, a device according to embodiments of the present disclosure may be dispensed from the carrier roll 40 by unrolling the material at least to reveal a first perforated line 42, and tearing the material from the roll at the perforated line for separating a single device from a remainder of the roll. A user may then manipulate or configure the device so as to reshape it from a generally flat state or configuration, as shown in FIG. 4, into a generally cylindrical state or configuration prior to use. In other embodiments, the body material of the device is initially formed with a hollow shape or profile, wherein the material's memory will act to return the device to a hollow state after being unraveled from the roll. In still other embodiments, additional perforations 42 may be formed through the material at a plurality of spaced intervals which are less than a typical desired length of the device (e.g., formed every 3 inches). In this way, a user may select a desired length of a device by tearing off a desired number of body segments depending on, for example, his or her height compared to the height of a toilet or other urine receptacle. As described above with respect to FIG. 3, a plurality of visual indication may be formed on the body in conjunction with or in place of the perforations 42. Such indications may include, for example, indications of length (e.g., 24″, 28″, 32″, etc.) printed on the body such that a user may quickly determine and appropriately size a device from a roll of material prior to use.

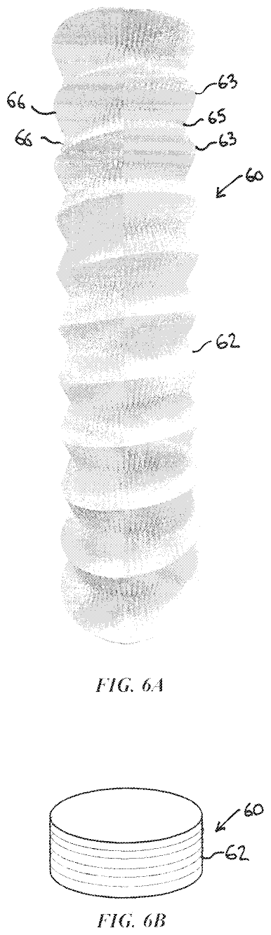

Referring to FIGS. 5 and 6A-6B, in other advantageous embodiments each of the devices includes a discrete body expandable in length from a compact position for storage, to an extended position for use. Specifically, referring to FIG. 5, a device 50 has a body 52 that has been rolled upon itself, in a manner similar to that of many male prophylactic devices. More specifically, starting at an output end 56 for example, the circumferential edge of the body 52 has been rolled on itself in a direction toward an input end 54, so as to define a circumferential roll. In order to utilize the device 50, a user may insert themselves into the input end 54, or otherwise align the input end 54 with their urethra, and unroll a remainder of the body 52 in the indicated direction until it is expanded to a desired or suitable length for a given toilet height.

Similarly, referring to FIGS. 6A and 6B, a device 60 includes a body 62 pre-formed so as to be foldable in an accordion-like manner. Specifically, the body 62 defines alternating circumferential folds 63,65 (i.e., folds having an apex extending radially outward alternating with folds having an apex extending radially inward). The alternating folds 63,65 define adjacent panels or pleats 66 of body material which are generally oriented parallel with one another in a stored or folded position, as shown in FIG. 6B, and generally axially aligned with one another in a fully expanded or extended position, with FIG. 6A illustrating a partially expanded positon. As with the embodiment of FIG. 5, in use, a user may only extend or expand the device 60 as far as necessary from the storage configuration, depending on their own height and the height of the toilet.

Accordingly, the embodiments of FIGS. 5 and 6A-6B provide a single device which is adjustable in length, compared to, for example, a device according to FIGS. 2-4, wherein a device length may be determined according to a selected number of segments separated from a continuous piece of material, or via the manual removal of material from a body prior to use. Moreover, as distinct from embodiments where the device is folded generally flat and must be expanded radially to use, by forming and storing the devices in either of the manners shown in FIGS. 5 and 6A-6B, devices may be more readily-usable, without requiring further manipulation to expand the devices into a hollow structure. It should be understood that either a constant-diameter device (e.g., device 20 of FIGS. 1 and 2), or a conical or tapering device (e.g., device 30 of FIG. 3) may be packaged or configured in any of the above-described manners.

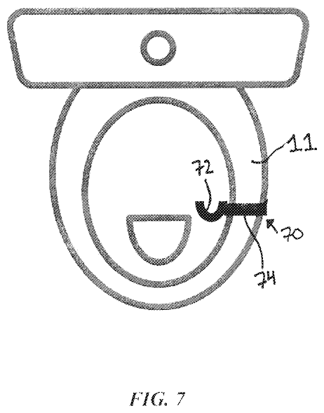

Referring now to FIG. 7, embodiments of the present disclose further include a locating bracket or guide 70 configured to be attached to a toilet bowl 11. The guide 70 defines a receiving aperture or guide surface into or through which an outlet end of the device may be associated. The guide 70 may include, for example, a hook-like or U-shaped clip 74 for securing about (i.e., on either side of) a rim of the toilet bowl 11, and a U-shaped or closed ring-shaped receiving guide surface 72 configured to receive and/or guide an outlet end of a urinary device according to embodiments of the present disclosure. In practice, a user may insert or engage the outlet end of the device with the bracket prior to use, ensuring the device is properly aligned with the toilet, further reducing the likelihood of accidental spillage. In some embodiments, the bracket may be configured to angle an outlet end of a device toward a wall of the toilet bowl, reducing both splashing and excess noise normally associated with expelled urine contacting the water.

While the foregoing invention has been described with reference to the above-described embodiment, various modifications and changes can be made without departing from the spirit of the invention. Accordingly, all such modifications and changes are considered to be within the scope of the appended claims. Accordingly, the specification and the drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations of variations of various embodiments. Combinations of the above embodiments, and other embodiments not specifically described herein, will be apparent to those of skill in the art upon reviewing the above description.

What is claimed is:

1. A method for aiding a user in urinating into a receptacle from a standing position, comprising:
configuring, by a user, a water-soluble urination device from a storage configuration to a use configuration wherein the device defines an elongated hollow body;
associating a first open end of the device with a urethra of the user;
placing a second open end of the device into water contained within the receptacle; and
with the device associated with the urethra of the user, guiding urine received in the first open end of the device to the second open end of the device and into the water.

2. The method of claim 1, wherein the device is formed from biodegradable paper.

3. The method of claim 1, wherein the step of configuring the device includes the further steps of:
unrolling the device from a continuous piece of material wound on a carrier roll, wherein the material defines a plurality of devices in the storage configuration; and
separating the device from a remainder of the material wound on the carrier roll.

4. The method of claim 3, wherein the step of separating the device includes tearing the material along a perforation.

5. The method of claim 1, the step of configuring the device includes the step of extending the second open end of the device relative to the first open end of the device in a longitudinal direction for increasing an overall length of the device

6. The method of claim 5, wherein the step of extending the second open end of the device relative to the first open end of the device includes unrolling a circumferential sidewall of the body.

7. The method of claim 5, wherein the step of extending the second open end of the device relative to the first open end of the device includes at least partially unfolding a plurality of circumferential folds formed in a sidewall of the body.

8. The method of claim 1, further comprising the step of placing a portion of the elongated hollow body onto a surface of a guide attached to the receptacle.

9. A sanitary urination device, comprising:
an elongated hollow body formed from a water soluble material and configured to convey urine from a first open end thereof to a second open end thereof.

10. The device of claim 9, wherein the material is a coated paper.

11. The device of claim 9, wherein the body includes a tubular shape having a continuous diameter from the first open end to the second open end.

12. The device of claim 11, further comprising a plurality of perforations formed through the body in a direction transverse to a direction of elongation of body.

13. The device of claim 9, wherein the body includes a diameter which tapers from the first open end thereof to the second open end thereof.

14. The device of claim 9, further comprising a carrier roll, wherein the device is wound around an exterior of the carrier roll.

15. The device of claim 14, wherein a plurality of devices are wound on the carrier rolled, the plurality of devices formed continuously with one another from a single piece of material.

16. The device of claim 15, wherein a plurality of perforations are formed through the material so as to delineate the plurality of devices wound on the carrier roll.

17. The device of claim 9, wherein the elongated body defines a plurality of circumferential folds.

18. The device of claim 17, wherein adjacent circumferential folds of the plurality of circumferential folds are oriented in opposite alternating radial directions, and wherein the device is extendable between a packaged configuration, wherein a plurality of pleats defined between the plurality of circumferential folds are arranged generally parallel with one another, and a use configuration, wherein the plurality of circumferential folds are at least partially unfolded for increasing an overall length of the device.
